(19) United States
(12) Patent Application Publication
(10) Pub. No.: US 2024/0426845 A1
(43) Pub. Date: Dec. 26, 2024

(54) METHOD FOR DETECTING A LIPIDATED PROTEIN VIA FLUORESCENCE RESONANCE ENERGY TRANSFER (FRET)

(71) Applicant: Biogen MA Inc., Cambridge, MA (US)

(72) Inventors: Kirk William Donovan, Cambridge, MA (US); Susanne Evelyn Swalley, Cambridge, MA (US); Xiaofeng Li, Belmont, MA (US)

(21) Appl. No.: 18/708,272

(22) PCT Filed: Nov. 15, 2022
(86) PCT No.: PCT/US2022/049925

§ 371 Date: May 8, 2024

Related U.S. Application Data
(60) Provisional application No. 63/279,885, filed on Nov. 16, 2021.

(51) Int. Cl. G01N 33/68; G01N 33/58; G01N 33/92

(52) U.S. Cl. CPC G01N 33/6896; G01N 33/582; G01N 33/92; G01N 2800/2821; G01N 2800/52

(57) ABSTRACT

Aspects of the present invention relate to methods for detecting a lipidated protein in a sample from a subject using a protein binding agent and a lipid binding agent configured for detecting the lipidated protein via a fluorescence resonance energy transfer (FRET) assay. In some aspects, such methods are useful for diagnosing and treating a subject such as a subject having a neurological disorder.

### CLAIM OF PRIORITY

This application claims the benefit of U.S. Provisional Patent Application No. 63/279,885, filed on Nov. 16, 2021, which is incorporated by reference herein in its entirety.

### BACKGROUND

Lipidation occurs in many proteins in eukaryotic cells and regulates numerous biological pathways, such as membrane trafficking, protein secretion, signal transduction, and apoptosis. Lipoproteins are complex particles with a central core containing cholesterol esters and triglycerides surrounded by free cholesterol, phospholipids, and apolipoproteins, which serve a structural role and facilitate lipoprotein formation. Apolipoproteins also act as ligands for lipoprotein receptors and serve as cofactors of enzymes involved in lipoprotein metabolism. Proteins can also be covalently modified by lipid molecules.

### SUMMARY

Provided herein are methods for detecting a lipidated protein in a quantitative manner that is rapid, cost-effective, normalizable, and amenable to high-throughput analysis. Detecting a lipidated protein, in some embodiments, is achieved using protein and lipid binding agents configured for detecting the lipidated protein via a fluorescence resonance energy transfer (FRET) assay.

Accordingly, aspects of the present disclosure provide a method for detecting a lipidated protein in a sample, the method comprising (a) providing a sample comprising a lipidated protein; (b) contacting the sample with a protein binding agent and a lipid binding agent under conditions in which the protein binding agent and the lipid binding agent are capable of binding to the lipidated protein, wherein the protein binding agent is labeled with a fluorophore donor and the lipid binding agent comprises an acceptor moiety, or vice versa; and (c) detecting a level of fluorescence resonance energy transfer (FRET) between the fluorophore donor and the acceptor moiety, wherein the level of FRET is indicative of a level of the lipidated protein in the sample.

In some embodiments, step (b) comprises incubating the sample with the protein binding agent prior to contacting the sample with the lipid binding agent. In some embodiments, the sample and the protein binding agent are incubated for at least 1 hour prior to contacting the sample with the lipid binding agent. In some embodiments, step (b) comprises incubating the sample with the lipid binding agent prior to contacting the sample with the protein binding agent.

In some embodiments, the protein binding agent is labeled with the acceptor moiety and the lipid binding agent the fluorophore donor. In some embodiments, the acceptor moiety is a fluorophore acceptor or a fluorescence quencher.

In some embodiments, the lipidated protein is a lipidated apolipoprotein. In some embodiments, the lipidated apolipoprotein is selected from the group consisting of apolipoprotein A-I (ApoA-I), apolipoprotein B (ApoB), apolipoprotein D (ApoD), an apolipoprotein E (ApoE), and apolipoprotein J (ApoJ). In some embodiments, the lipidated apolipoprotein is an ApoE. In some embodiments, the ApoE is ApoE2, ApoE3, or ApoE4.

In some embodiments, the protein binding agent is a protein, a nucleic acid, or a small molecule. In some embodiments, the protein binding agent is an antibody or an antigen binding fragment thereof.

In some embodiments, the lipidated apolipoprotein is an ApoE and the protein binding agent is an anti-ApoE antibody or an antigen binding fragment thereof. In some embodiments, the lipidated apolipoprotein is ApoE2 and the protein binding agent is an anti-ApoE2 antibody or an antigen binding fragment thereof. In some embodiments, the lipidated apolipoprotein is ApoE3 and the protein binding agent is an anti-ApoE3 antibody or an antigen binding fragment thereof. In some embodiments, the lipidated apolipoprotein is ApoE4 and the protein binding agent is an anti-ApoE4 antibody or an antigen binding fragment thereof.

In some embodiments, the anti-ApoE antibody or antigen binding fragment thereof is the antibody produced by clone EPR19392, clone E6D7, or clone WUE-4, or an antigen binding fragment thereof.

In some embodiments, the lipid binding agent is a lipophilic fluorescent dye. In some embodiments, the lipophilic fluorescent dye is selected from the group consisting of a lipophilic carbocyanine dye, a lipophilic aminostyryl dye, a PKH dye, and a lipophilic styryl dye. In some embodiments, the lipophilic carbocyanine dye is selected from the group consisting of Dil, DiO, DiD, and DiR. In some embodiments, the lipophilic aminostyryl compound is DiA or an analog thereof. In some embodiments, the PKH dye is PKH2, PKH26, or PKH67. In some embodiments, the lipophilic styryl dye is FM® 1-43, FM® 1-43FX, FM® 2-10, FM® 4-64, FM® 4-64FX, or FM® 5-95.

In some embodiments, the lipid binding agent comprises a fluorophore or a fluorescent quencher. In some embodiments, the fluorophore donor is a lanthanide metal. In some embodiments, the lanthanide metal is europium, terbium, or samarium.

In some embodiments, the sample is a cell culture. In some embodiments, the sample is a biological sample obtained from a subject. In some embodiments, the biological sample comprises blood, serum, plasma, or cerebral spinal fluid (CSF).

In some embodiments, the subject has a neurological disorder. In some embodiments, the neurological disorder is a neurodegenerative disease selected from the group consisting of Alzheimer's disease (AD), multiple sclerosis (MS), and Parkinson's disease (PD). In some embodiments, the neurological disorder is a neurological injury selected from the group consisting of a traumatic brain injury (TBI), a spinal cord injury (SCI), and a stroke. In some embodiments, the neurological disorder is selected from the group consisting of epilepsy, dementia, and meningoencephalitis.

Aspects of the present disclosure provide a method for treating a subject having a neurological disorder, the method comprising: (a) detecting the level of the lipidated protein in the sample according to the methods described herein; (b) comparing the level of the lipidated protein in the sample to a reference level; and (c) administering a treatment if the level of the lipidated protein in the sample is equal to or lower than the reference level.

In some embodiments, the treatment is selected from the group consisting of a lipid modifying agent, an immunosuppressive therapy, a cholinesterase inhibitor, an anti-seizure therapy, an anti-inflammatory therapy, dopamine, a dopamine agonist, an antibiotic, an antiviral therapy, an apolipoprotein targeted therapy, or a combination thereof.

In some embodiments, the lipid modifying agent is an ABCA1 agonist or a nuclear receptor agonist. In some embodiments, the ABCA1 agonist is an antisense oligonucleotide or a peptide. In some embodiments, the antisense oligonucleotide is an antisense oligonucleotide targeting miR-33 or an antisense oligonucleotide targeting ARF6. In some embodiments, the peptide is selected from the group consisting of CS-6253, Ac-hE18A-NH2, and 4F. In some embodiments, the nuclear receptor agonist is a LXR agonist or a RXR agonist. In some embodiments, the LXR agonist is TO901317 or GW3965. In some embodiments, the RXR agonist is bexarotene, LG100268, SPF1, or SPF2.

In some embodiments, the apolipoprotein targeted therapy is an ApoE targeted therapy selected from the group consisting of an anti-ApoE antibody or an antigen binding fragment thereof (e.g., an anti-ApoE antibody or an antigen binding fragment thereof binds to unlipidated ApoE), a nucleic acid that inhibits expression of ApoE (e.g., a siRNA targeting ApoE or an antisense oligonucleotide targeting ApoE), an agent that blocks the activity of and/or lowers the level of ApoE (e.g., an agent that blocks the activity of and/or lowers the level of unlipidated ApoE), an ApoE4 structure corrector, an anti-ApoE4 immunotherapy, an agent that enhances expression of ApoE2, and agent that inhibits expression of ApoE4. In some embodiments, the ApoE4 structure corrector is PH002, GIND105, or GIND-25. In some embodiments, the anti-ApoE4 immunotherapy is the anti-human ApoE4 antibody HAE-4. In some embodiments, the agent that enhances expression of ApoE2 is an AAV-expressing human ApoE2. In some embodiments, the agent that inhibits expression of ApoE4 is an antisense oligonucleotide targeting ApoE4. In some embodiments, the anti-ApoE antibody or an antigen binding fragment thereof binds to unlipidated ApoE. In some embodiments, the nucleic acid that inhibits expression of ApoE is an antisense oligonucleotide targeting ApoE or an siRNA targeting ApoE. In some embodiments, the agent that blocks the activity of and/or lowers the level of ApoE blocks the activity of and/or lowers the level of unlipidated ApoE.

In some embodiments, the neurological disorder is Alzheimer's disease. In some embodiments, the lipidated protein is ApoE4. In some embodiments, the treatment is an anti-amyloid beta antibody. In some embodiments, the anti-amyloid beta antibody is aducanumab, bapineuzumab, gantenerumab, solanezumab, donanemab, or lecanemab.

### DETAILED DESCRIPTION OF THE DRAWINGS

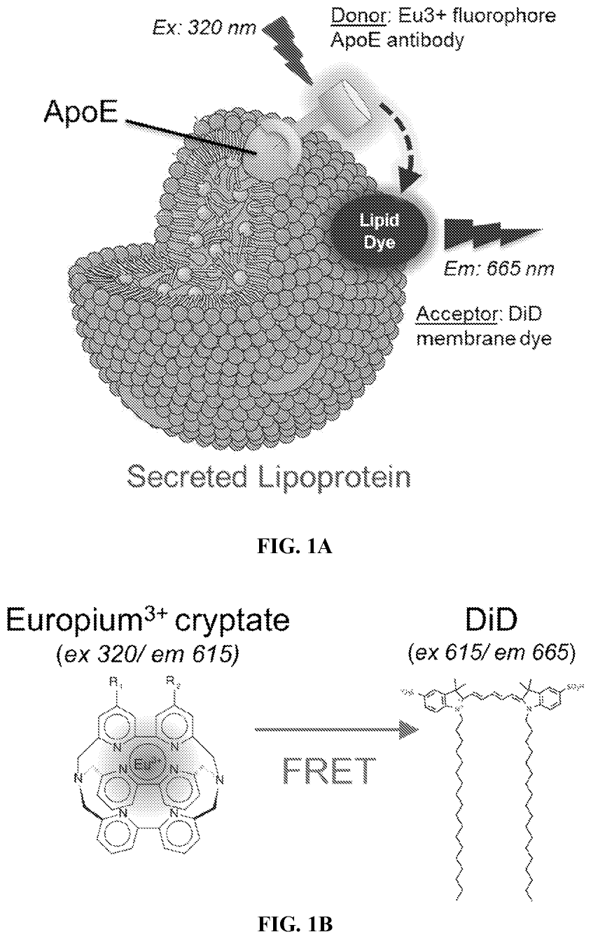

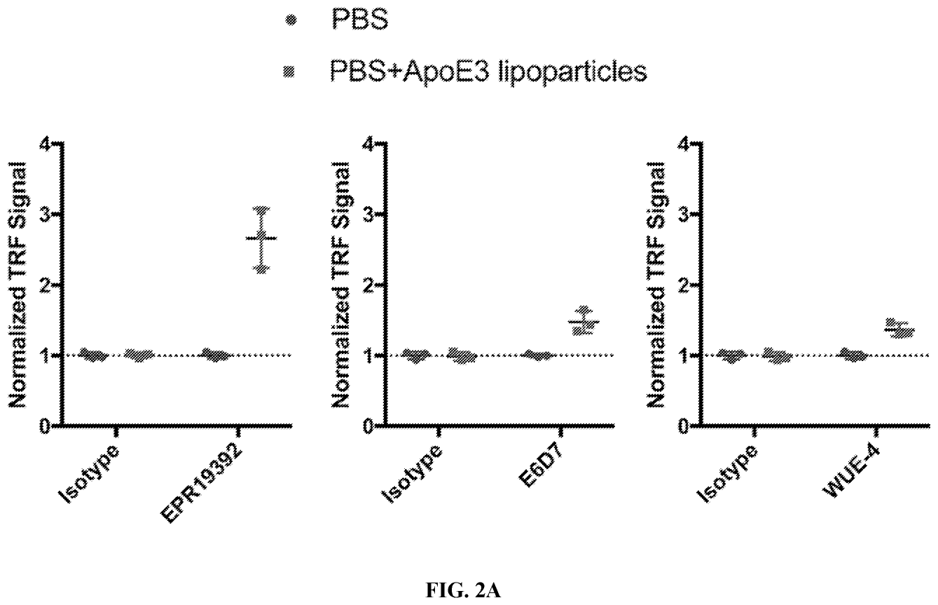

FIG. 1A is a schematic diagram of TR-FRET between a europium-conjugated anti-ApoE antibody and lipid dye DiD on a secreted lipoprotein particle. DiD inserts itself into the lipids while the europium-conjugated antibody binds to ApoE. When the pair are in close proximity and the antibody is excited with 320 nm light, an energy transfer from the antibody donor occurs that can be detected by an increase in emitted 665 nm light that is emitted from the FRET acceptor.

FIG. 1B is a schematic diagram of structures of europium cryptate FRET donor and DiD FRET acceptor. Fluorescence excitation and emission values for the pair are also shown.

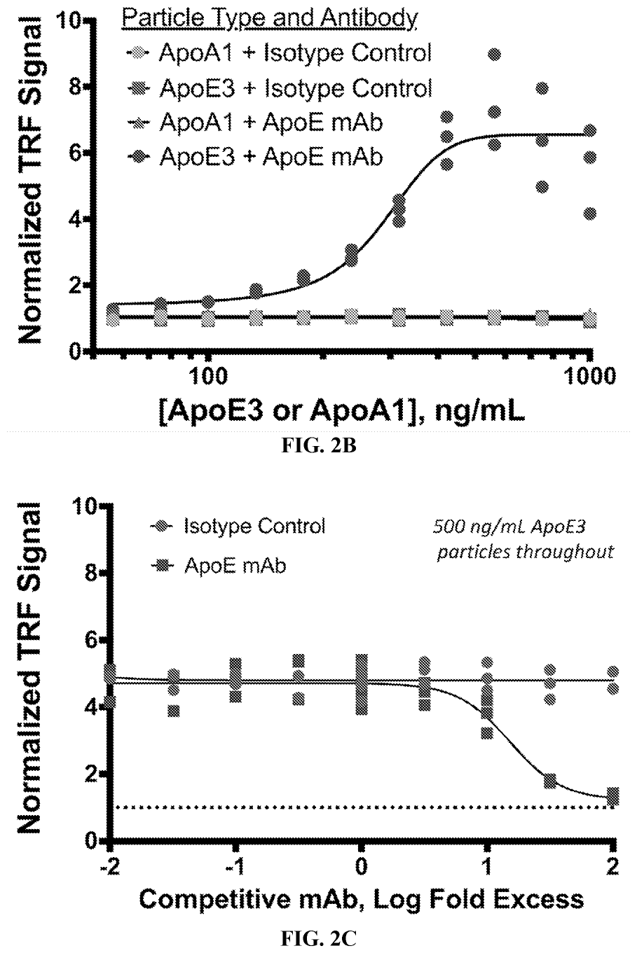

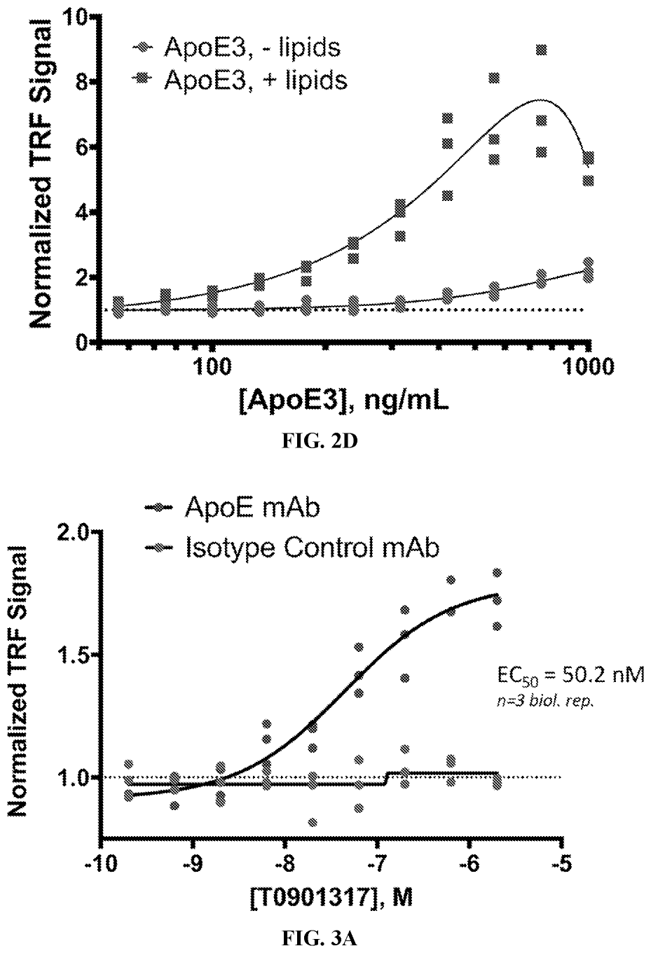

FIG. 2A is a graph of results from TR-FRET ApoE lipidation assays performed with several europium-conjugated ApoE or isotype control antibodies and in vitro generated ApoE3 lipoprotein particles. A TR-FRET signal detecting ApoE lipoprotein particles was observed for all three different ApoE antibodies (EPR19392, E6D7, and WUE-4).

FIG. 2B is a graph of results from TR-FRET ApoE lipidation assays performed using in vitro generated ApoE3 lipoprotein particles or ApoA1 particles. The TR-FRET signal was specific for ApoE3 lipoprotein particles detected with an anti-ApoE antibody. n=3 biological replicates.

FIG. 2C is a graph of results from TR-FRET ApoE lipidation assays performed using in vitro generated ApoE3 lipoprotein particles and increasing concentrations of unlabeled ApoE or isotype control antibody. The reduction in signal of the ApoE mAb curve is due to the unlabeled anti-ApoE antibody competing for binding to the ApoE epitope in place of the europium-labeled anti-ApoE antibody, which disrupts the TR-FRET signal and indicates that the assay is specific for ApoE lipoprotein particles. n=3 biological replicates.

FIG. 2D is a graph of results from TR-FRET ApoE lipidation assays performed using in vitro generated ApoE3 particles (+lipids) or soluble lipid-free ApoE (− lipids). The TR-FRET signal was specific for lipidated ApoE particles. n=3 biological replicates.

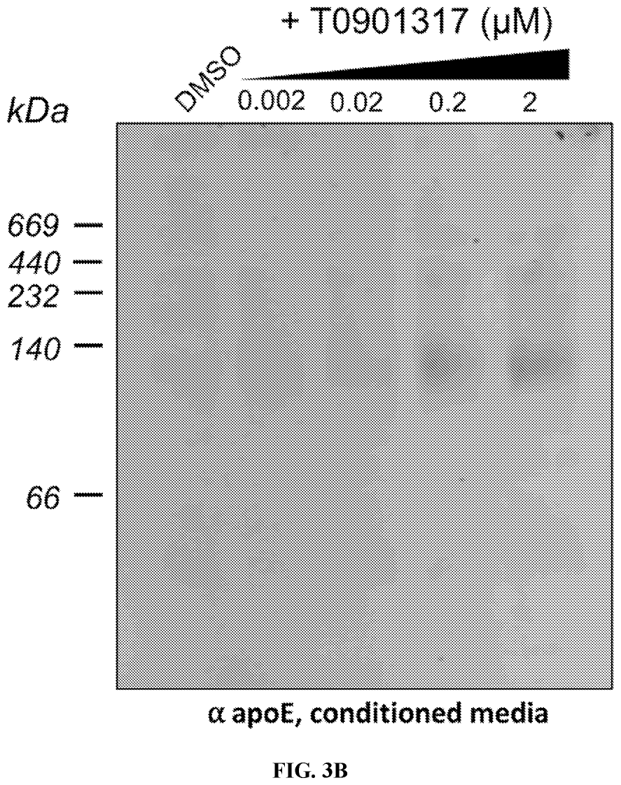

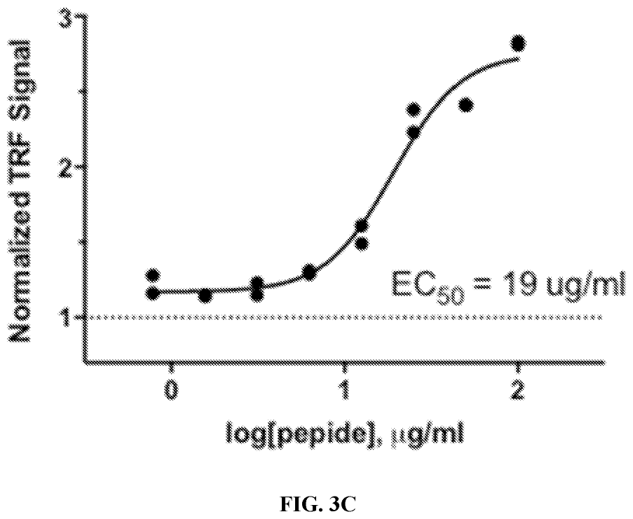

FIG. 3A is a graph of results from TR-FRET ApoE lipidation assays performed using media from CCF-STTG cells dosed with T0901317, a LXR transcription factor agonist that induces ApoE lipoprotein secretion. T0901317 was incubated with cells for 48 hours before media collection. The FRET signal showed a concentration-dependent response and was only detectable with a europium-conjugated anti-ApoE antibody. EC₅₀ for T0901317 was 50.2 nM. n=3 biological replicates.

FIG. 3B is an image of a native-PAGE gel showing ApoE lipidation in conditioned media. The conventional method of detecting ApoE lipidation is native-PAGE gel, which is limited by low sample throughput and variation between gels. The EC₅₀ for ApoE lipidation observed from the native-PAGE gel was similar to that obtained using the TR-FRET assay.

FIG. 3C is a graph of results from a TR-FRET ApoE lipidation assay performed using media from BIONi010 ApoE3/3 iPSC-derived astrocytes treated with a peptide (e.g., an ABCA1 peptide agonist). The EC₅₀ for this peptide agonist is 19 μg/mL.

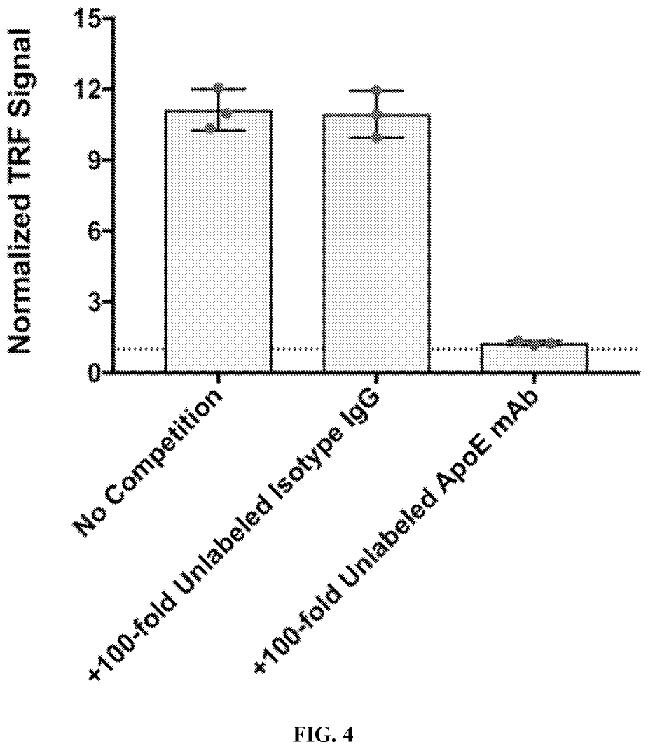

FIG. 4 is a graph of results from TR-FRET ApoE lipidation assays performed using human CSF. Human CSF was incubated with either 100-fold excess of an unlabeled isotype control antibody or 100-fold excess of unlabeled anti-ApoE antibody. The reduction in signal is due to the unlabeled anti-ApoE antibody binding to the ApoE epitope in place of the europium-labeled anti-ApoE antibody, which disrupts the TR-FRET signal and indicates that the assay is specific for ApoE lipoprotein particles in human CSF. n=3 biological replicates.

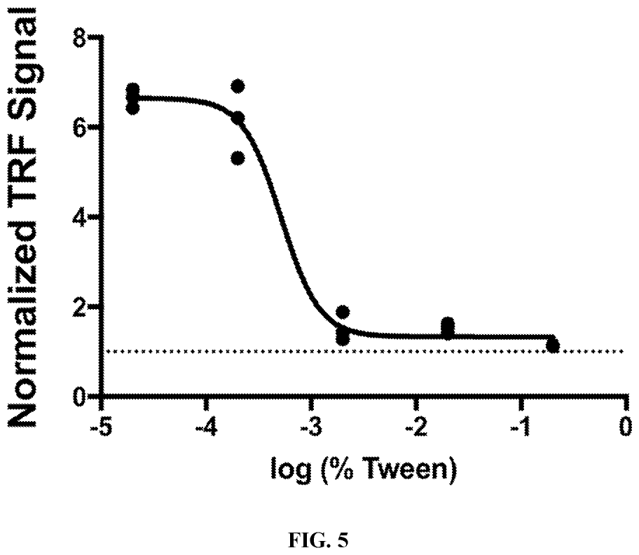

FIG. 5 is a graph of results from TR-FRET ApoE lipidation assays performed using human CSF in the presence of the detergent Tween-20. Human CSF was incubated with low concentrations (sub-critical micelle concentration) of Tween-20. TR-FRET signal was reduced at the higher concentrations, likely due to the detergent stripping lipids in the lipoprotein particle away from ApoE and disturbing FRET pair interaction, which indicates that the assay is specific for ApoE lipoprotein particles in human CSF (EC₅₀=0.00052% Tween-20). n=3 biological replicates.

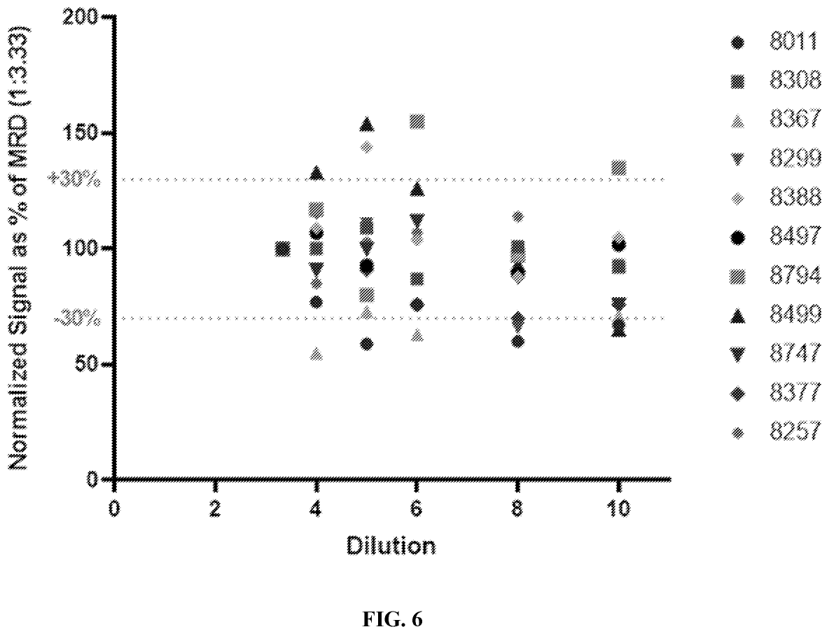

FIG. 6 is a graph of results from testing linearity of the TR-FRET signal from endogenous lipidated ApoE using human CSF. Samples of CSF from Alzheimer's disease patients were serial diluted in artificial CSF matrix and assayed for ApoE lipidation. Normalized lipidated ApoE signal was linear within at least two dilutions beyond the minimum required dilution (MRD) in 8 of 11 of samples tested.

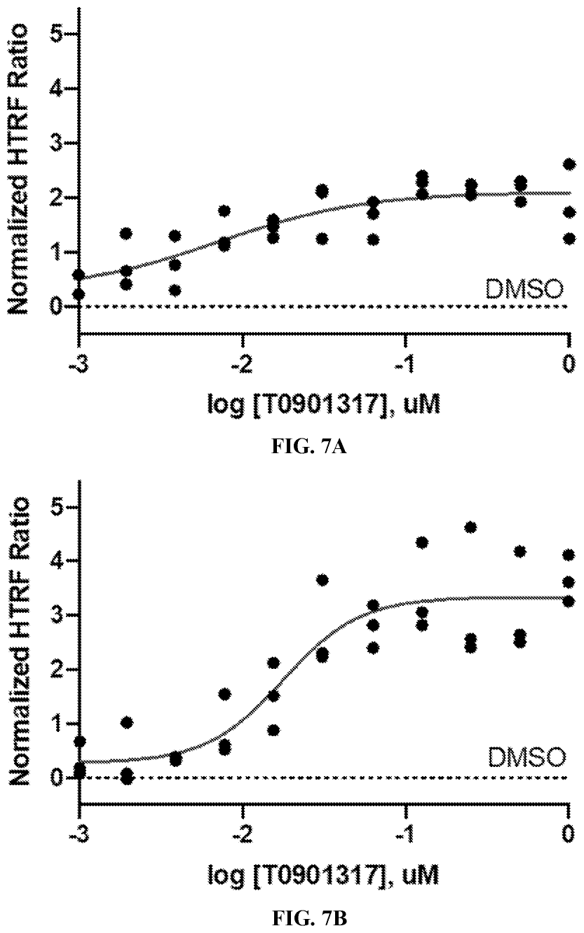

FIG. 7A is a graph of results from TR-FRET ApoE lipidation assays performed using co-addition of anti-ApoE antibody and DiD to samples. Experiments were performed using media from CCF-STTG cells dosed with T0901317.

FIG. 7B is a graph of results from TR-FRET ApoE lipidation assays performed using sequential addition of anti-ApoE antibody and DiD to samples. Experiments were performed using media from CCF-STTG cells dosed with T0901317.

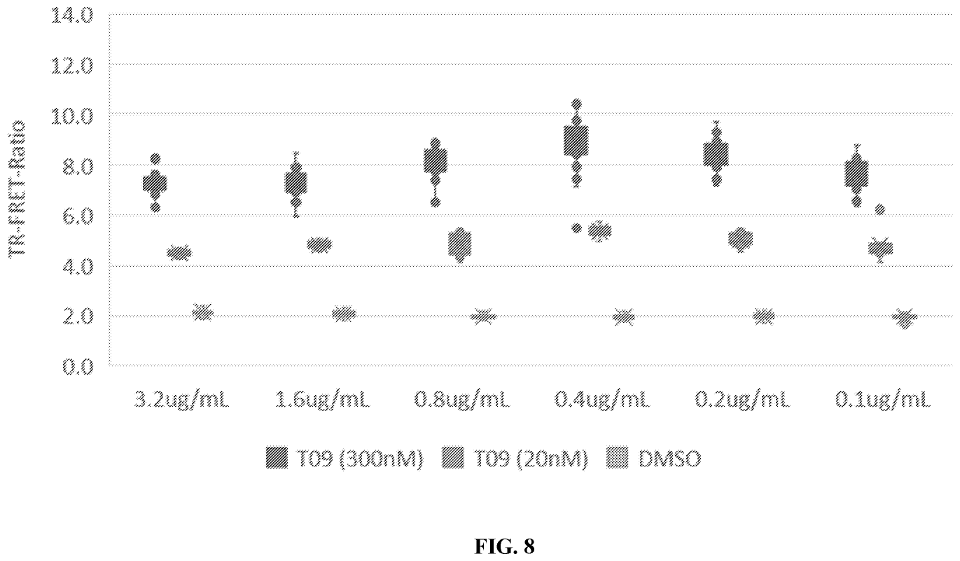

FIG. 8 is a graph of results from TR-FRET ApoE lipidation assays performed using increasing concentrations of DiD. Experiments were performed using media from CCF-STTG cells dosed with T0901317 or DMSO as a control.

### DETAILED DESCRIPTION

The present disclosure is based, at least in part, on the development of a fluorescence resonance energy transfer (FRET) assay for detecting lipidated proteins. Methods described herein are designed for specifically detecting lipidated proteins in a biological sample. The level of lipidated protein is often indicative of status of diseases associated with biological pathways in which lipids and/or the protein of interest play a role. As such, methods described herein are useful in diagnosis and prognosis of diseases mediated by lipidation, e.g., neurological diseases and disorders.

### I. Assay Components

The FRET assay methods described herein involve a protein binding agent and a lipid binding agent, each of which is labeled with one member of a FRET pair.

### (a) Protein Binding Agents

A protein binding agent is a molecule (e.g., a small molecule, a protein such as an antibody, a nucleic acid such as an aptamer) that specifically binds a protein of interest. In some examples, the protein binding agent is specific to a lipidated protein of interest. In other examples, the protein binding agent binds to both lipidated and non-lipidated forms of the protein of interest.

The protein binding agent is said to exhibit “specific binding” if it reacts more frequently, more rapidly, with greater duration and/or with greater affinity with a particular protein (e.g., those disclosed herein) than it does with other molecules. “Specific binding” or “preferential binding” does not necessarily require (although it can include) exclusive binding.

The protein binding agent for use in methods disclosed herein may have any binding affinity suitable for binding and detecting a lipidated protein. As used herein, “binding affinity” refers to the apparent association constant or KA. The KA is the reciprocal of the dissociation constant (KD). The antibody described herein may have a KD of at least 10⁻⁵, 10⁻⁶, 10⁻⁷, 10⁻⁸, 10⁻⁹, 10⁻¹⁰ M, or lower. An increased binding affinity corresponds to a decreased KD.

The protein binding agent can be an antibody that binds a target protein. As used herein, the term “antibody” refers to a protein that includes at least one immunoglobulin variable domain or immunoglobulin variable domain sequence. For example, an antibody can include a heavy (H) chain variable region (VH) and a light (L) chain variable region (VL). In another example, an antibody includes two heavy chain variable regions and two light chain variable regions. The term “antibody” encompasses full-length antibodies and antigen-binding fragments of antibodies (e.g., single chain antibodies, Fab and sFab fragments, F(ab′)₂, Fd fragments, Fv fragments, scFv, and domain antibodies (dAb) fragments. An antibody can have the structural features of IgA, IgG, IgE, IgD, IgM (as well as subtypes thereof).

In some embodiments, an antibody that binds a lipidated protein may specifically bind to the lipidated protein, for example, an epitope of the lipidated protein. An antibody that “specifically binds” to an antigen or an epitope is a term well understood in the art, and methods to determine such specific binding are also well known in the art. An antibody is said to exhibit “specific binding” if it reacts or associates more frequently, more rapidly, with greater duration and/or with greater affinity with a particular target than it does with alternative targets. An antibody “specifically binds” to a target antigen or epitope if it binds with greater affinity, avidity, more readily, and/or with greater duration than it binds to other molecules. For example, an antibody that specifically (or preferentially) binds to an antigen (e.g., a lipidated apolipoprotein such as ApoE) or an antigenic epitope therein is an antibody that binds this target antigen with greater affinity, avidity, more readily, and/or with greater duration than it binds to other antigens or other epitopes in the same target antigen. It is also understood that an antibody that specifically binds to a first target antigen may or may not specifically or preferentially bind to a second target antigen. As such, “specific binding” or “preferential binding” does not necessarily require (although it can include) exclusive binding. Generally, but not necessarily, reference to binding means preferential binding. In some examples, an antibody that “specifically binds” to a target antigen or an epitope thereof may not bind to other antigens or other epitopes in the same target antigen.

An antibody that binds a lipidated protein can be either monoclonal or polyclonal. A “monoclonal antibody” refers to a homogenous antibody population and a “polyclonal antibody” refers to a heterogeneous antibody population. These two terms do not limit the source of an antibody or the manner in which it is made. An antibody for use in methods disclosed herein can be commercially obtained or made by any method known in the art, e.g., conventional hybridoma technology.

In some embodiments, the protein binding agent binds a human lipidated apolipoprotein. For example, when the human lipidated apolipoprotein is ApoE (e.g., ApoE2, ApoE3, or ApoE4), the protein binding agent can be an anti-ApoE antibody or antigen binding fragment thereof (e.g., an anti-ApoE2 antibody or fragment thereof, an anti-ApoE3 antibody or fragment thereof, an anti-ApoE4 antibody or fragment thereof).

An anti-ApoE antibody or antigen binding fragment thereof can bind to one isoform of ApoE (referred to as isoform specific antibodies) or multiple isoforms of ApoE (referred to as pan-ApoE antibodies). For example, the anti-ApoE antibody or antigen binding fragment thereof can specifically bind to a single isoform of ApoE, e.g., the anti-ApoE antibody or antigen binding fragment thereof binds ApoE2 but does not bind to ApoE3 or ApoE4. An example anti-ApoE antibody that binds to a single isoform of ApoE is provided in clone 4E4, which specifically binds to ApoE4. Anti-ApoE4 antibody prepared from clone 4E4 is commercially available, e.g., from Thermo Fisher Scientific, Abcam, and Novus Biologicals.

In some examples, the anti-ApoE antibody or antigen binding fragment thereof can bind to multiple isoforms of ApoE, e.g., the anti-ApoE antibody or antigen binding fragment thereof binds ApoE2, ApoE3, and ApoE4. Example anti-ApoE antibodies that bind to multiple isoforms of ApoE are provided in clone EPR19392, clone E6D7, and clone WUE-4. Antibodies that bind to ApoE are commercially available, e.g., from Abcam, Novus Biologicals, Santa Cruz Biotechnology, R&D Systems, and Invitrogen.

### (b) Lipid Binding Agents

A lipid binding agent is a molecule (e.g., a small molecule, a protein such as an antibody, a nucleic acid such as an aptamer). The lipid binding agent is said to exhibit “specific binding” if it reacts more frequently, more rapidly, with greater duration and/or with greater affinity with lipids than it does with other molecule(s).

The lipid binding agent can be intrinsically fluorescent (e.g., a lipophilic fluorescent dye) or extrinsically fluorescent (e.g., a lipid binding agent labeled with a fluorophore). In some examples, the lipid binding agent is non-fluorescent (e.g., a lipid binding agent labeled with a fluorescence quencher).

Non-limiting examples of a lipophilic fluorescent dye include a lipophilic styryl dye (e.g., FM® dyes such as FM® 1-43, FM® 1-43FX, FM® 2-10, FM® 4-64, FM® 4-64FX, or FM® 5-95), lipophilic carbocyanine dye (e.g., Dil, DiO, DiD, or DiR), a lipophilic aminostyryl dye (e.g., DiA), or a PKH dye (e.g., PKH2, PKH26, or PKH67).

### (c) FRET Pairs

A “FRET pair” consists of a fluorophore donor and an acceptor moiety such as a fluorophore acceptor or a fluorescence quencher. Any FRET pair (e.g., fluorophore donor and fluorophore acceptor) suitable for FRET can be used in methods disclosed herein. FRET pairs for use in methods described herein are known in the art and commercially available, e.g., from Life Technologies (Carlsbad, CA), GE Healthcare (Piscataway, NJ), Integrated DNA Technologies (Coralville, IA), and Roche Applied Science (Indianapolis, IN).

As used herein, a “fluorophore donor” refers to a fluorophore that, upon absorbing light, can transfer excitation energy to a fluorophore acceptor or a fluorescence quencher. Non-limiting examples of a fluorophore donor include Alexa Fluor 488, Alexa Fluor 568, Alexa Fluor 594, Alexa Fluor 647, Cy2, Cy3, BODIPY, GFP, fluorescein, IEDANS, EDANS, or a lanthanide metal (e.g., europium, terbium, samarium).

As used herein, a “fluorophore acceptor” refers to a fluorophore that can accept excitation energy transferred by a fluorophore donor and use the transferred energy to emit light at its own characteristic emission wavelength spectrum. Non-limiting examples of a fluorophore acceptor include Cy3, Cy5, R-Phycoerythrin (R-PE), allophycocyanin (APC), Alexa Fluor 532, Alexa Fluor 546, Alexa Fluor 610, Alexa Fluor 647, BODIPY, fluorescein, and YFP.

As used herein, a “fluorescence quencher” refers to a non-fluorescent molecule that can accept energy from an excited fluorophore, thereby reducing the fluorescence signal of the fluorophore. Non-limiting examples of a fluorescence quencher include Dabcyl, Tamra, and Black Hole Quenchers.

It should be understood that a fluorophore can be a fluorophore donor when paired with one fluorophore and it can be a fluorophore acceptor when paired with another fluorophore. For example, Cy3 is a fluorophore donor when paired with Cy5, and Cy3 is a fluorophore acceptor when paired with Cy2.

### II. Detection of Lipidated Proteins

Also provided herein are methods for detecting lipidated proteins (e.g., lipidated apolipoprotein such as ApoE) in a sample. The assay methods disclosed herein involve the use of a protein binding agent and a lipid binding agent, both of which are disclosed herein. The protein binding agent is labeled with a fluorophore donor and the lipid binding agent is labeled with a fluorophore acceptor or vice versa (i.e., the protein binding agent is labeled with a fluorophore acceptor and the lipid binding agent is labeled with a fluorophore donor).

To perform the assay methods disclosed herein, a sample suspected of containing lipidated protein can be brought in contact with the protein binding agent and the lipid binding agent under conditions suitable for formation of a complex comprising the lipidated protein, the protein binding agent, and the lipid binding agent, if any. The presence or level of the lipidated protein in the sample can be detected by measuring a FRET signal generated when the fluorophore donor is excited by light and in close proximity to the fluorophore acceptor. Detection can be at a wavelength of the emission spectrum of the fluorophore donor, the fluorophore acceptor, or both.

As used herein, the term “contacts” refers to an exposure of a sample with a protein binding agent and a lipid binding agent for a time and under conditions sufficient for the formation of a complex with a lipidated protein in the sample, if any.

In some examples, assay methods can be performed using a fluorophore donor and a fluorescence quencher. In such instances, a wavelength of the emission spectrum of the fluorophore donor can be detected, and the fluorescence readout will be reduced with increased binding of the protein and lipid binding agents to the lipidated protein, which brings the fluorescence quencher and the fluorophore donor into proximity with one another.

In some examples, FRET measurements are time-resolved. Measurement of time-resolved fluorescence resonance energy transfer can reduce the interference from background fluorescence.

Methods for detecting a lipidated protein provided herein, in some embodiments, comprise (a) providing a sample comprising a lipidated protein; (b) contacting the sample with a protein binding agent and a lipid binding agent under conditions in which the protein binding agent and the lipid binding agent are capable of binding to the lipidated protein, wherein the protein binding agent is labeled with one member of a fluorophore donor and acceptor pair and the lipid binding agent is labeled with the other member of the fluorophore donor and acceptor pair; and (c) detecting a level of FRET between the fluorophore donor and the fluorophore acceptor, wherein the level of FRET is indicative of a level of the lipidated protein in the sample.

In some embodiments, methods for detecting a lipidated protein comprise (a) contacting the sample with a protein binding agent and a lipid binding agent under conditions in which the protein binding agent and the lipid binding agent are capable of binding to the lipidated protein, wherein the protein binding agent is labeled with one member of a fluorophore donor and acceptor pair and the lipid binding agent is labeled with the other member of the fluorophore donor and acceptor pair; and (b) detecting a level of FRET between the fluorophore donor and the fluorophore acceptor, wherein the level of FRET is indicative of a level of the lipidated protein in the sample.

Methods provided herein encompass contacting the sample with the protein binding agent and the lipid binding agent, e.g., simultaneously or sequentially. In some embodiments, methods provided herein comprise contacting the sample with the protein binding agent and the lipid binding agent simultaneously. In other embodiments, methods provided herein encompass contacting the sample with either one of the protein binding agent or the lipid binding agent and then incubating the sample for a period of time suitable to allow formation of a complex between the lipidated protein and the protein binding agent or the lipid binding agent, if any. Following the incubation period, the sample can be contacted with the other agent (i.e., the lipid binding agent or the protein binding agent) to form a three-component complex that includes the lipidated protein, the protein binding agent, and the lipid binding agent.

For example, if the sample was incubated with the protein binding agent, then the sample can be contacted with the lipid binding agent after the incubation period. Alternatively, if the sample was incubated with the lipid binding agent, then the sample can be contacted with the lipid binding agent after the incubation period.

Without wishing to be bound by theory, incubating the sample with either one of the protein binding agent or the lipid binding agent and then incubating the sample with the other agent can increase complex formation between the lipidated protein, the protein binding, and the lipid binding agent and/or increase level of FRET signal between the fluorophore donor and the acceptor moiety.

Accordingly, methods for detecting a lipidated protein provided herein, in some embodiments, comprise (a) contacting the sample with a protein binding agent and incubating the sample under conditions in which the protein binding agent is capable of binding to the lipidated protein, wherein the protein binding agent is labeled with one member of a fluorophore donor and acceptor pair; (b) contacting the sample with a lipid binding agent, wherein the lipid binding agent is labeled with the other member of the fluorophore donor and acceptor pair; and (c) detecting a level of FRET between the fluorophore donor and the fluorophore acceptor, wherein the level of FRET is indicative of a level of the lipidated protein in the sample.

In some embodiments, methods for detecting a lipidated protein provided herein, in some embodiments, comprise (a) contacting the sample with a lipid binding agent and incubating the sample under conditions in which the lipid binding agent is capable of binding to the lipidated protein, wherein the lipid binding agent is labeled with one member of a fluorophore donor and acceptor pair; (b) contacting the sample with a protein binding agent, wherein the protein binding agent is labeled with the other member of the fluorophore donor and acceptor pair; and (c) detecting a level of FRET between the fluorophore donor and the fluorophore acceptor, wherein the level of FRET is indicative of a level of the lipidated protein in the sample.

In some examples, the sample and one of the protein binding agent or the lipid binding agent can be incubated for 5 minutes to 2 hours prior to contacting the sample with the other agent. For example, the sample and the protein binding agent or the lipid binding agent can be incubated for 10 minutes to 2 hours, 20 minutes to 2 hours, 30 minutes to 2 hours, 40 minutes to 2 hours, 50 minutes to 2 hours, 1 hour to 2 hours, 1.5 hours to 2 hours, 5 minutes to 1.5 hours, 5 minutes to 1 hour, 5 minutes to 50 minutes, 5 minutes to 40 minutes, 5 minutes to 30 minutes, 5 minutes to 20 minutes, 5 minutes to 15 minutes, or 5 minutes to 10 minutes.

In some examples, after incubating the sample and one of the protein binding agent or the lipid binding agent, the other agent can be contacted with the sample, and then the sample can be incubated with both agents for 1 to 48 hours, e.g., 1 to 36 hours, 1 to 24 hours, 1 to 12 hours, 1 to 6 hours, 6 to 48 hours, 12 to 48 hours, 24 to 48 hours, or 36 to 48 hours.

For example, methods described herein can comprise incubating the sample and the protein binding agent (e.g., an antibody such as an anti-ApoE antibody such as clone EPR19392) for 5 minutes to 2 hours (e.g., 1 hour), contacting the sample with the lipid binding agent (e.g., a lipophilic fluorescent dye such as DiD), and incubating the sample comprising the protein binding agent and the lipid binding agent for 1 to 48 hours (e.g., 20 hours).

In another example, methods described herein can comprise incubating the sample and the lipid binding agent (e.g., a lipophilic fluorescent dye such as DiD) for 5 minutes to 2 hours (e.g., 1 hour), contacting the sample with the protein binding agent (e.g., an antibody or an antigen binding fragment thereof such as an anti-ApoE antibody or an antigen binding fragment thereof such as clone EPR19392 or an antigen binding fragment of clone EPR19392), and incubating the sample comprising the protein binding agent and the lipid binding agent for 1 to 48 hours (e.g., 20 hours).

A sample comprising the protein binding agent and/or the lipid binding agent can be incubated at any temperature suitable for detection of a lipidated protein. For example, the sample can be incubated at a temperature between 25° C. and 40° C., e.g., at a temperature of 25° C., 26° C., 27° C., 28° C., 29° C., 30° C., 31° C., 32° C., 33° C., 34° C., 35° C., 36° C., 37° C., 38° C., 39° C., or 40° C.

Any amount of protein binding agent suitable for detection of a lipidated protein in a sample can be used in methods described herein. In some examples, the amount of protein binding agent (e.g., antibody) in the sample is between 0.05 to 1 μg/mL, e.g., 0.1 to 1 μg/mL, 0.25 to 1 μg/mL, 0.5 to 1 μg/mL, 0.75 to 1 μg/mL, 0.05 to 0.75 μg/mL, 0.05 to 0.05 μg/mL, 0.05 to 0.25 μg/mL, or 0.05 to 0.1 μg/mL. In some examples, the amount of protein binding agent (e.g., antibody) in the sample is between 0.05 μg/mL, 0.1 μg/mL, 0.25 μg/mL, 0.5 μg/mL, 0.75 μg/mL, or 1 μg/mL.

Any amount of lipid binding agent suitable for detection of a lipidated protein in a sample can be used in methods described herein. In some examples, the amount of lipid binding agent (e.g., DiD) in the sample is between 50 to 5000 ng/mL, e.g., 250 to 5000 ng/mL, 500 to 5000 ng/mL, 750 to 5000 ng/mL, 1000 to 5000 ng/mL, 2000 to 5000 ng/mL, 3000 to 5000 ng/mL, 4000 to 5000 ng/mL, 50 to 4000 ng/mL, 50 to 3000 ng/mL, 50 to 2000 ng/mL, 50 to 1000 ng/mL, 50 to 750 ng/mL, 50 to 500 ng/mL, or 50 to 250 ng/mL. In some examples, the amount of lipid binding agent (e.g., DiD) in the sample is 50 ng/mL, 250 ng/mL, 500 ng/mL, 1000 ng/mL, 2000 ng/mL, 3000 ng/mL, 4000 ng/mL, or 5000 ng/mL, 50 to 3000 ng/mL, 50 to 2000 ng/mL, 50 to 1000 ng/mL, 50 to 750 ng/mL, 50 to 500 ng/mL, or 50 to 250 ng/mL. In some examples, the amount of DiD in the sample is 50 ng/mL, 250 ng/mL, 500 ng/mL, 1000 ng/mL, 2000 ng/mL, 3000 ng/mL, 4000 ng/mL, or 5000 ng/mL, 50 to 3000 ng/mL, 50 to 2000 ng/mL, 50 to 1000 ng/mL, 50 to 750 ng/mL, 50 to 500 ng/mL, or 50 to 250 ng/mL.

In some examples, the amount of lipid binding agent (e.g., DiD) in the sample is between 50 to 500 ng/mL, e.g., 100 to 500 ng/mL, 150 to 500 ng/mL, 200 to 500 ng/mL, 250 to 500 ng/mL, 300 to 500 ng/mL, 350 to 500 ng/mL, 400 to 500 ng/mL, 450 to 500 ng/mL, 50 to 450 ng/mL, 50 to 400 ng/mL, 50 to 350 ng/mL, 50 to 300 ng/mL, 50 to 250 ng/mL, 50 to 200 ng/mL, 50 to 150 ng/mL, or 50 to 150 ng/mL. In some embodiments, the amount of lipid binding agent (e.g., DiD) in the sample is 50 ng/mL, 100 ng/mL, 150 ng/mL, 200 ng/mL, 250 ng/mL, 300 ng/mL, 350 ng/mL, 400 ng/mL, 450 ng/mL, or 500 ng/mL. In some embodiments, the amount of DiD in the sample is 50 ng/mL, 100 ng/mL, 150 ng/mL, 200 ng/mL, 250 ng/mL, 300 ng/mL, 350 ng/mL, 400 ng/mL, 450 ng/mL, or 500 ng/mL.

Methods provided herein encompass detecting a lipidated protein, or lack thereof, in various samples. In some embodiments, the sample is a biological sample obtained from a subject. In some embodiments, the biological sample is obtained from a subject having (or at risk of having) a neurological disorder. In some embodiments, the biological sample comprises blood, serum, plasma, cerebral spinal fluid (CSF), or a combination thereof. In some embodiments, the biological sample is concentrated prior to use in any of the methods described herein. In some embodiments, the biological sample is not concentrated prior to use in any of the methods described herein.

Any lipidated protein can be detected using methods described herein. In some examples, the lipidated protein is a protein involved in lipid management (e.g., metabolism, synthesis, transport). Such proteins include, but are not limited to, apolipoproteins (e.g., ApoA-I, ApoB, ApoD, ApoE, ApoJ), lipoproteins (e.g., ApoB100), phospholipid transfer proteins (PLTPs), fatty acid-binding proteins (FABPs), and LDL receptors (LDLRs). In some examples, the lipidated protein is a protein involved in synaptic vesicle trafficking (e.g., alpha-synuclein). In some examples, the lipidated protein is a transcription factor (e.g., a transcriptional enhanced associate domain (TEAD) transcription factor). In some examples, the lipidated protein is a chaperone (e.g., Hsp70). In some examples, the lipidated protein is an oncoprotein (e.g., a Ras protein, a Wnt protein, a Src kinase). In some examples, the lipidated protein is a pathogenic protein (e.g., a viral protein, a parasitic protein).

### II. Applications of FRET Assays

Methods described herein can be applied to evaluating a disease, e.g., diagnosis and/or prognosis of a disease. Evaluation can include identifying a subject as being at risk for or having a disease, e.g., a neurological disorder. Evaluation can also include monitoring treatment of a disease such as evaluating the effectiveness of a treatment for a disease. Examples of a neurological disorder include, but are not limited to, a neurodegenerative disease (e.g., Alzheimer's disease (AD), multiple sclerosis (MS), Parkinson's disease (PD)) and a neurological disorder such as epilepsy, dementia, meningoencephalitis, or a neurological injury (e.g., a traumatic brain injury (TBI), a spinal cord injury (SCI), a stroke).

### (a) Diagnosis

Methods described herein can be used to determine the level of the lipidated protein in a sample (e.g., a blood sample, a serum sample, a plasma sample, a CSF sample) collected from a subject, and the level of the lipidated protein is then compared to a reference level to determine whether the subject has or is at risk for a disease, e.g., a neurological disorder. In some examples, the disease (e.g., prior to onset of symptoms) is diagnosed if the level of lipidated protein in the sample from the subject is lower than a control level. In other examples, the disease (e.g., prior to onset of symptoms) is diagnosed if the level of lipidated protein in the sample from the subject is higher than a control level.

By comparing the level of lipidated protein in a sample obtained from a subject to the reference level as described herein, it can be determined as to whether the subject has or is at risk for a disease. For example, if the level of lipidated protein in the sample from the subject is reduced as compared to the reference value, the subject is identified as having or at risk for a neurological disorder. The assay disclosed herein can be used to predetermine a cutoff value representing lipidated protein levels in normal subjects. Such a cutoff value can be used for determining whether a subject has or is at risk for a disease. In some instances, the level of lipidated protein in a subject is below the cutoff value may indicate disease risk or occurrence.

As used herein, “a decreased level or a level below a reference value” means that the level of lipidated protein is lower than a reference value, such as a pre-determined threshold or a level of lipidated protein in a control sample.

A decreased level of lipidated protein includes a lipidated protein level that is, for example, 50%, 60%, 70%, 80%, 90%, 100%, 150%, 200%, 300%, 400%, 500% or more lower than a reference value. In some embodiments, a decreased level of lipidated protein includes a lipidated protein level that is at least 1.1, 1.2, 1.3, 1.4, 1.5, 1.6, 1.7, 1.8, 1.9, 2, 2.5, 3, 3.5, 4, 4.5, 5, 6, 7, 8, 9, 10, 25, 50, 75, 100, 150, 200, 300, 400, 500, 1000-fold or more lower than the level of the reference level. A decreased level of lipidated protein also includes decreasing a phenomenon from a non-zero state (e.g., some or detectable lipidated protein in a sample) to a zero state (e.g., no or undetectable lipidated protein in a sample).

In some embodiments, the subject is a human patient having a symptom of a neurological disorder. For example, the subject has persistent or sudden onset of headaches, loss of feeling or tingling, weakness or loss of muscle strength, blurry vision or double vision, memory loss, impaired mental ability, lack of coordination, or combinations thereof. In some embodiments, the subject has no symptom of a neurological disorder at the time the sample is collected, has no history of a symptom of a neurological disorder, or no history of a neurological disorder.

In some examples, if the level of lipidated protein in the sample from the subject is increased as compared to the reference value, the subject is identified as having or at risk for a disease (e.g., hyperlipidemia). For example, if the level of lipidated protein in the sample from the subject is elevated as compared to the reference value, the subject is identified as having or at risk for a disease.

As used herein, “an elevated level or a level above a reference value” means that the level of lipidated protein is higher than a reference value, such as a pre-determined threshold or a level of lipidated protein in a control sample.

An elevated level of lipidated protein includes a lipidated protein level that is, for example, 50%, 60%, 70%, 80%, 90%, 100%, 150%, 200%, 300%, 400%, 500% or more above than a reference value. In some embodiments, an elevated level of lipidated protein includes a lipidated protein level that is at least 1.1, 1.2, 1.3, 1.4, 1.5, 1.6, 1.7, 1.8, 1.9, 2, 2.5, 3, 3.5, 4, 4.5, 5, 6, 7, 8, 9, 10, 25, 50, 75, 100, 150, 200, 300, 400, 500, 1000-fold or more higher than the level of the reference level. An elevated level of lipidated protein also includes increasing a phenomenon from a zero state (e.g., no or undetectable lipidated protein in a sample) to a non-zero state (e.g., some or detectable lipidated protein in a sample).

### (b) Treatment and Treatment Effectiveness

Aspects of the present disclosure provide treatment methods involving detection of the level of the lipidated protein in a sample according to any of the methods described herein.

In some embodiments, methods described herein can comprise (a) detecting the level of the lipidated protein in the sample according to any of the methods described herein; (b) comparing the level of the lipidated protein in the sample to a reference level; and (c) administering a treatment if the level of the lipidated protein in the sample deviates from the reference level (e.g., the level of the lipidated protein is equal to or lower than the reference level; or the level of the lipidated protein is equal to or greater than the reference level).

For example, when a subject has or is at risk for Alzheimer's disease, methods described herein can comprise detecting the level of lipidated ApoE4 in a sample from a patient using any of the methods described herein, comparing the level of lipidated ApoE4 in the sample to a reference level, and administering a treatment to the patient if the level of the lipidated ApoE4 in the sample is equal to or lower than the reference level. Such methods encompass treating the patient with any therapeutic agent known in the art or described herein, e.g., an anti-amyloid beta antibody such as aducanumab, bapineuzumab, gantenerumab, solanezumab, donanemab, or lecanemab.

Methods described herein can also be applied to evaluate the effectiveness of a treatment for a neurological disorder. For example, multiple biological samples (e.g., blood, serum, plasma, or CSF samples) can be collected from a subject to whom a treatment is performed either before and after the treatment or during the course of the treatment. The levels of lipidated protein can be measured by any method described herein. For example, if the level of the lipidated protein increases after the treatment or over the course of the treatment (the level of lipidated protein in a later collected sample as compared to that in an earlier collected sample), remains the same or increases, it indicates that the treatment is effective.

If the subject is identified as not responsive to the treatment, a higher dose and/or frequency of dosage of the therapeutic agent are administered to the subject identified. In some embodiments, the dosage or frequency of dosage of the therapeutic agent is maintained, lowered, or ceased in a subject identified as responsive to the treatment or not in need of further treatment. Alternatively, a different treatment can be applied to the subject who is found as not responsive to the first treatment.

If the level of the lipidated protein in the sample from the subject deviates from the reference level (e.g., is lower than the reference level or is higher than the reference level), the subject can be administered a lipid modifying agent. For example, when the reference level is the level of lipidated protein in a sample from a healthy subject, if the level of the lipidated protein in the sample from a subject having a neurological disorder deviates from the reference level, the subject can be administered a lipid modifying agent.

In some examples, if the level of the lipidated protein in the sample from the subject is equal to the reference level, the subject can be administered a lipid modifying agent. For example, when the reference level is the level of lipidated protein in a sample from a first subject having a neurological disorder, if the level of the lipidated protein in the sample from a second subject having a neurological disorder is equal to the reference level, the second subject can be administered a lipid modifying agent.

As used herein, a “lipid modifying agent” refers to any agent that is capable of modifying a level of a lipid (e.g., the level of the lipid in the blood and/or the liver of the subject). Lipid modifying agents encompass agents that lower lipid levels (e.g., lipid lowering agents) as well as agents that increase lipid levels (e.g., lipid enhancing agents). Any lipid modifying agent known in the art can be used in methods described herein.

Non-limiting examples of a lipid modifying agent include an ABCA1 agonist, a nuclear receptor agonist, vitamin E supplementation, a statin (e.g., atorvastatin), a bile-acid-binding resin (e.g., cholestyramine), and a cholesterol absorption inhibitor (e.g., asezetimibe).

In some examples, the ABCA1 agonist is an antisense oligonucleotide (e.g., an antisense oligonucleotide targeting miR-33 or an antisense oligonucleotide targeting ARF6) or a peptide (e.g., CS-6253, Ac-hE18A-NH2, 4F). In some examples, the nuclear receptor agonist is a LXR agonist (e.g., TO901317, GW3965) or a RXR agonist (e.g., bexarotene, LG100268, SPF1, SPF2). See, e.g., Lanfranco et al., Int. J Mol. Sci. 2020, 21, 6336, which is incorporated by reference for the purposes and subject matter referenced herein.

Any of the detection methods described herein can further comprise administering a therapy to a subject. Methods for evaluating treatment effectiveness described herein can be applied to any treatment suitable for use in a subject (e.g., a subject having a neurological disorder described herein).

Treatments for neurological disorders can vary depending on the condition. Non-limiting examples of treatments for a neurological disorder include immunosuppressive therapies, cholinesterase inhibitors, anti-seizure therapies, anti-inflammatory therapies, dopamine, dopamine agonists, antibiotics, antiviral therapies, and apolipoprotein targeted therapies.

Treatments for neurological disorders for use in methods described herein encompass treatment with agents that target unlipidated proteins such as apolipoproteins. For example, the treatment can comprise a nucleic acid that inhibits expression of the protein (e.g., an antisense oligonucleotide targeting the protein or an siRNA targeting the protein). In another example, the treatment can comprise an antibody or an antigen binding fragment thereof that binds to the unlipidated form of the target protein.

As used herein, an “apolipoprotein targeted therapy” refers to any agent that is capable of modifying a biological activity and/or a level of an unlipidated apolipoprotein (e.g., the level of expression of an unlipidated apolipoprotein) and/or a biological activity and/or a level of lipidated apolipoprotein. Apolipoprotein targeted therapies encompass agents that target ApoA, ApoB, ApoC, ApoD, ApoE or a combination thereof.

Any apolipoprotein targeted therapy can be used in methods described herein. Non-limiting examples of apolipoprotein targeted therapies include volanesorsen (targeting ApoC-III; see, e.g., Witztum et al., N Engl J Med 2019; 381:531-542), pelacarsen (targeting ApoA; see, e.g., Hardy et al., Am J Cardiovasc Drugs 2021, doi.org/10.1007/s40256-021-00499-1), and mipomersen (targeting ApoB; see, e.g., Santos et al., Arterioscler Thromb Vasc Biol 2015 35(3):689-699).

In some embodiments, the apolipoprotein targeted therapy is an antibody or an antigen binding fragment thereof capable of binding to an unlipidated apolipoprotein, e.g., any antibody or an antigen binding fragment thereof such as those known in the art or described herein. In some embodiments, the apolipoprotein targeted therapy is a nucleic acid targeting an unlipidated apolipoprotein, e.g., a small interfering RNA (siRNA) or an antisense oligonucleotide targeting an unlipidated apolipoprotein (e.g., ApoA-I, ApoB, ApoD, ApoE, ApoJ).

In some examples, the apolipoprotein targeted therapy is an ApoE targeted therapy. As used herein, an “ApoE targeted therapy” refers to any agent that is capable of modifying a biological activity and/or a level of unlipidated ApoE (e.g., the level of expression of ApoE) and/or a biological activity and/or a level of lipidated ApoE. ApoE targeted therapies encompass agents that target ApoE2, ApoE3, ApoE4, or a combination thereof.

Non-limiting examples of an ApoE targeted therapy include a structure corrector (e.g., ApoE4 structure corrector), an anti-ApoE immunotherapy (e.g., an anti-ApoE4 antibody), an agent that enhances expression of ApoE (e.g., ApoE2), and an agent that inhibits expression of ApoE (e.g., ApoE4).

In some embodiments, the ApoE targeted therapy comprises an antibody or an antigen binding fragment thereof that is capable of binding ApoE (e.g., ApoE2, ApoE3, ApoE4, or a combination thereof). In some embodiments, the ApoE targeted therapy comprises an siRNA targeting ApoE (e.g., ApoE2, ApoE3, ApoE4, or a combination thereof) or an antisense oligonucleotide targeting ApoE (e.g., ApoE2, ApoE3, ApoE4, or a combination thereof).

In some examples, the ApoE4 structure corrector is PH002, GIND105, or GIND-25. In some examples, the anti-ApoE4 immunotherapy is the anti-human ApoE4 antibody HAE-4. In some examples, the agent that enhances expression of ApoE2 is an AAV-expressing human ApoE2. In some examples, the agent that inhibits expression of ApoE4 is a siRNA targeting ApoE4 or an antisense oligonucleotide targeting ApoE4.

In some examples, ApoE targeted therapy includes editing of an ApoE gene, which can be achieved using any method known in the art such as CRISPR-Cas9 gene editing technologies.

### (c) Controls

Methods described herein involve determining the level of a lipidated protein in a sample from a subject, wherein an elevated level of lipidated protein in the sample compared to a reference level predicts whether a subject is likely to develop a neurological disease. In some embodiments, the control level is a level of lipidated protein in a control sample that is capable of binding to a protein and lipid binding agent. In some embodiments, a control sample is obtained from a healthy subject or population of healthy subjects. As used herein, a healthy subject is a subject that is apparently free of a neurological disorder at the time the level of lipidated protein is measure or a subject that has no history of the disorder. The control level as described herein can be determined by methods described herein or by methods known in the art.

The control level can also be a predetermined level. The predetermined level or score can be a single cut-off (threshold) value, such as a median or mean, or a level or score that defines the boundaries of an upper or lower quartile, tertile, or other segment of a population that is determined to be statistically different from the other segments. It can be a range of cut-off (or threshold) values, such as a confidence interval. It can be established based upon comparative groups, such as where association with risk of developing disease or presence of disease in one defined group is a fold higher, or lower, (e.g., approximately 2-fold, 4-fold, 8-fold, 16-fold or more) than the risk or presence of disease in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

The predetermined value can depend upon the particular population of subjects (e.g., human subjects) selected. For example, an apparently healthy population will have a different ‘normal’ range of levels or scores than will a population of subjects which have, are likely to have, or are at greater risk to have, a disorder described herein. Accordingly, the predetermined values selected may take into account the category (e.g., sex, age, health, risk, presence of other diseases) in which a subject (e.g., human subject) falls. Appropriate ranges and categories can be selected with no more than routine experimentation by those of ordinary skill in the art.

In some embodiments, the predetermined level or score is a level or score determined in the same subject, e.g., at a different time point, e.g., an earlier time point. In characterizing likelihood, or risk, numerous predetermined values can be established.

Accordingly, method described herein include determining if the level of lipidated protein falls above or below a predetermined cut-off value.

A cut-off value is typically a lipidated protein above or below which is considered predictive of something, e.g., likely to develop a neurological disorder or responsiveness of a subject to a therapy. Thus, in accordance with the methods described herein, a reference level of lipidated protein is identified as a cut-off value, above or below of which is predictive of a subject being likely to develop a neurological disorder. Some cut-off values are not absolute in that clinical correlations can still remain significant over a range of values on either side of the cutoff; however, it is possible to select an optimal cut-off value (e.g., varying H-scores) of the level of lipidated protein for a particular sample type. Cut-off values determined for use in the methods described herein can be compared with, e.g., published ranges of levels of lipidated protein, but can be individualized to the methodology used and patient population. It is understood that improvements in optimal cut-off values could be determined depending on the sophistication of statistical methods used and on the number and source of samples used to determine reference level values for the different lipidated proteins and sample types. Therefore, established cut-off values can be adjusted up or down, on the basis of periodic re-evaluations or changes in methodology or population distribution.

### Examples

In order that the invention described may be more fully understood, the following examples are set forth. The examples described in this application are offered to illustrate the methods and compositions provided herein and are not to be construed in any way as limiting their scope.

### Materials and Methods

The following materials and methods were used in the Examples set forth herein.

### Preparation of ApoE3 Lipoprotein Particles

ApoE3 lipoprotein particles for proof-of-principle in vitro experiments were prepared by first adding 40 mg DMPC lipid (dimyristoylphosphatidylcholine, Avanti #850345) into a small glass beaker containing 10 mL of PBS (4 mg/mL solution). DMPC was allowed to hydrate for 10 minutes at room temperature before sonicating on ice using a fine-tipped sonicator at 50% power for 1 hour. The opaque solution was spun at 10,000×g for 5 minutes before the supernatant was transferred to a fresh tube on ice. Recombinant ApoE3 protein (Peprotech, #350-02) was reconstituted at 2 mg/mL in a 20 mM Na₂HPO₄ (pH 7.8) buffer. 150 mM β-mercaptoethanol was added to the rehydrated ApoE3 and incubated at room temperature for 30 min. Finally, the sonicated DMPC vesicles were added to the ApoE3 protein solution at a ratio of 3.75 mg vesicles per 1 mg ApoE3 and the mixture was incubated at room temperature for 3 hours. The ApoE:DMPC lipoprotein complexes then went through 3 cycles of heating and cooling (4° C. to 37° C.) before use.

### Europium-Conjugated Antibodies

Custom europium-conjugated antibodies were purchased from CisBio. Anti-ApoE antibody clones EPR19392 (Abcam #ab227993), E6D7 (Abcam #ab1907), and WUE-4 (BioRad #MCA5639), rabbit IgG isotype control (Thermo #02-6102), and mouse IgG isotype control (Thermo #02-6102) were labeled with approximately 5-6 europium chelates per antibody.

### Human Cerebrospinal Fluid (CSF)

Human CSF was purchased from BioIVT. Total protein concentration was 3 mg/mL as determined by ELISA.

### 1,1′-dioctadecyl-3,3,3′,3′-tetramethylindodicarbocyanine, 4-chlorobenzenesulfonate salt (“DiD”)

DiD was purchased from Invitrogen (cat. no. D7757). A stock solution of 10 mg/mL DiD in DMSO was prepared and stored at −20° C. Working aliquots of 1 mg/mL DiD in DMSO were stored at room temperature in the dark.

### FRET Analysis

TR-FRET analysis was performed on an Envision plate reader (Perkin Elmer). The donor fluorophore (europium cryptate-conjugated antibody) was excited at 320 nm. A 40 μs delay occurred before measuring emission signals at 620 nm (donor fluorescence) and 665 nm wavelengths (DiD acceptor FRET signal) during a 400 μs window. Sample normalized FRET signal was determined by finding the ratio of the 665 nm emission to 620 nm emission.

### Example 1: Overview of a TR-FRET Assay for Detection of Lipidated Proteins

A schematic depiction of a time-resolved fluorescence resonance energy transfer (TR-FRET) assay for detecting lipidated proteins such as apolipoprotein E (ApoE) is shown in FIG. 1A. ApoE is the main lipid carrier in the brain and must be secreted and properly lipidated to take part in various normal cellular processes including neuronal growth, repair and remodeling of membranes, synaptogenesis, clearance and degradation of amyloid β (Aβ), and reducing neuroinflammation.

To detect lipidated ApoE using the TR-FRET assay, ApoE proteins in ApoE lipoprotein particles are recognized and bound by an anti-ApoE antibody conjugated to a europium cryptate, which acts as a fluorophore donor. Lipid dyes such as DiD are inserted into the lipids of the lipoprotein and act as a fluorophore acceptor. When the fluorophore donor (europium) is excited by light and in close proximity (e.g., about 4 to 8 nm) to the fluorophore acceptor (lipid dye), a detectable FRET signal is generated that indicates lipidated ApoE is detected. Structures of the europium cryptate and DiD are shown in FIG. 1B.

### Example 2: TR-FRET Assay Detects Lipidated Proteins Generated In Vitro

To test whether the TR-FRET assay detects in vitro lipidated proteins, experiments were performed using in vitro generated apolipoprotein particles. ApoE3 lipoprotein particles (or PBS) were mixed with buffer, DiD, and one of several europium-conjugated ApoE antibodies or europium-conjugated control antibodies of the same isotype. As shown in FIG. 2A, the TR-FRET signal for ApoE lipoparticles was detectable for the three ApoE antibodies tested.

To determine the specificity between apoliprotein particles, increasing amounts of ApoA1 particles and ApoE3 particles were mixed with buffer, DiD and either a europium-conjugated isotype control antibody or a europium-conjugated anti-ApoE antibody (EPR19392 was used). As shown in FIG. 2B, the TR-FRET signal was detectable and specific for samples containing the anti-ApoE antibody and ApoE3 particles. As shown in FIG. 2C, specificity of the TR-FRET signal was confirmed by outcompeting europium-conjugated anti-ApoE antibody with unlabeled anti-ApoE antibody. Detection of lipid-free and particle bound apolipoprotein was tested by mixing europium-conjugated anti-ApoE antibody and DiD with increasing amounts of free ApoE3 or ApoE3 particles. As shown in FIG. 2D, the TR-FRET signal was specific for detection of ApoE3 particles, not free unlipidated ApoE3.

These results demonstrate that TR-FRET between a labeled protein binding agent and a labeled lipid binding agent is detectable and specific for lipoprotein particles generated in vitro.

### Example 3: TR-FRET Assay Detects Lipidated Proteins Secreted from Cells

To test whether the TR-FRET assay detects lipidated proteins secreted from cells, the TR-FRET assay was tested on concentrated media from cultures of CCF-STTG1 astrocytoma cells grown in the absence and presence of T0901317, a LXR transcription factor agonist that induces ApoE lipoprotein secretion. Samples of concentrated media were mixed with TR-FRET buffer, europium-conjugated anti-ApoE3 antibody and DiD, incubated at 37° C. for 20 hours, and then fluorescence was measured using an Envision plate reader. As a control, samples were analyzed by native-PAGE gels, a conventional method of detecting lipidated proteins. As shown in FIG. 3A, ApoE lipoprotein particles secreted in media from CCF-STTG1 cells dosed with T0901317 was detected in a dose-dependent manner using DiD and europium conjugated anti-ApoE antibody, but not the isotype control antibody. As shown in FIG. 3B, the presence of dose-dependent accumulation of lipidated ApoE in cell media following culture with T0901317 was confirmed using native-PAGE. Additionally, as shown in FIG. 3C, ApoE lipoprotein particles secreted from ApoE3/3 iPSC-derived astrocytes could be detected in a dose dependent manner using the TR-FRET method when the cells were treated with an exemplary ABCA1 peptide agonist (e.g., a peptide believed to increase ABCA1-mediated cholesterol efflux and apo-protein lipidation) (see, e.g., Hafiane et al. PLoS One. 10(7):2015).

These results demonstrate that TR-FRET between a labeled protein binding agent and a labeled lipid binding agent is detectable and specific for lipidated proteins secreted from cells.

### Example 4: TR-FRET Assay Detects Lipidated Proteins in Human CSF

To test whether the TR-FRET assay detects lipidated proteins in human CSF, the TR-FRET assay was tested on commercially available human CSF samples. Human CSF was thawed on ice and diluted in PBS (3 parts CSF to 7 parts PBS). 8 μL of diluted sample, buffer, europium-conjugated ApoE3 antibody (final antibody concentration of 0.2 μg/mL), and DiD (final concentration of 10 μg/mL) were added per well of the 384 well plate. Unlabeled isotype control antibody and unlabeled anti-ApoE antibody were used as controls. As shown in FIG. 4, lipidated ApoE was detected in human CSF using europium-conjugated anti-ApoE antibody and DiD. The specificity of lipidated ApoE detection was shown by a drastic reduction of the signal in the presence of excess unlabeled anti-ApoE antibody but not excess unlabeled isotype control antibody. Specificity of the assay is also shown in FIG. 5, where the TR-FRET assay was performed with human CSF and low concentrations of the detergent Tween-20. TR-FRET signal is reduced at the higher concentrations, likely due to the detergent stripping lipids in the lipoprotein particle away from ApoE and disturbing the FRET pair interaction.

The linearity of the TR-FRET signal from endogenous lipidated ApoE was evaluated in human CSF to characterize matrix interference and selectivity. Linearity was tested by serial dilution of CSF in artificial CSF matrix prior to evaluation in the assay. In CSF from both healthy control (data not shown) and Alzheimer's disease CSF (FIG. 6), normalized lipidated ApoE signal was linear, with at least two dilutions beyond the minimum required dilution (MRD) (1:3.33) resulting in signal within 30% of the signal detected at the MRD in over 70% of CSF samples tested (8 of 11).

These results demonstrate that TR-FRET between a labeled protein binding agent and a labeled lipid binding agent is detectable and specific for lipidated proteins found in human CSF.

### Example 5: Pre-Incubation of Sample and Antibody Improves Detection of Lipidated Proteins

To test whether pre-incubation of a sample with a protein binding agent improves detection of lipidated proteins, protein binding agent and lipid binding agent were sequentially added or co-added to samples of media (media not concentrated prior to use) from cultures of CCF-STTG1 astrocytoma cells grown in the absence or presence of increasing amounts of T0901317 to induce ApoE lipoprotein secretion from the cells. Samples of media were mixed with TR-FRET buffer, 0.2 μg/mL of europium-conjugated anti-ApoE3 antibody EPR19392, and then incubated at 37° C. for 1 hour. After incubation, 200 ng/mL of DiD was added to the samples, the samples were incubated at 37° C. for 20 hours, and then fluorescence was measured using an Envision plate reader. Control samples were prepared by co-addition of 0.2 μg/mL of europium-conjugated anti-ApoE3 antibody EPR19392 and 200 ng/mL of DiD to samples, and then incubating the samples at 37° C. As shown in FIGS. 7A-7B, samples in which antibody and DiD were added sequentially (FIG. 7B) had improved signal compared to samples in which antibody and DiD were co-added (FIG. 7A).

These results demonstrate that pre-incubation of a sample with a protein binding agent (also referred to as sequential addition of protein binding agent and lipid binding agent) can improve the detectable signal in the TR-FRET assay.

### Example 6: Increasing the Signal of the TR-FRET Assay

To test whether the signal of the TR-FRET assay could be increased, varying amounts of lipid binding agent were added to samples of concentrated media from cultures of CCF-STTG1 astrocytoma cells grown in the absence or presence of T0901317 to induce ApoE lipoprotein secretion from the cells. As a control, cells were incubated with DMSO. Samples of concentrated media were mixed with TR-FRET buffer, 0.2 μg/mL of europium-conjugated anti-ApoE3 antibody EPR19392, and then incubated at 37° C. for 1 hour. After incubation, DiD at the selected concentration (3.2 μg/mL, 1.6 μg/mL, 0.8 μg/mL, 0.4 μg/mL, 0.2 μg/mL, or 0.1 μg/mL) was added, samples were incubated at 37° C. for 20 hours, and then fluorescence was measured using an Envision plate reader. As shown in FIG. 8, higher signals were produced when the concentration of DiD was between 0.8 μg/mL to 0.2 μg/mL.

These results demonstrate that the concentration of lipid binding agent is not linear with the signal produced in the TR-FRET assay, and therefore higher signals in the TR-FRET assay can be achieved using lower concentrations of lipid binding agent.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for detecting a lipidated protein in a sample, the method comprising:
(a) providing a sample comprising a lipidated protein;
(b) contacting the sample with a protein binding agent and a lipid binding agent under conditions in which the protein binding agent and the lipid binding agent are capable of binding to the lipidated protein, wherein the protein binding agent is labeled with a fluorophore donor and the lipid binding agent comprises an acceptor moiety, or vice versa; and
(c) detecting a level of fluorescence resonance energy transfer (FRET) between the fluorophore donor and the acceptor moiety, wherein the level of FRET is indicative of a level of the lipidated protein in the sample.

2. The method of claim 1, wherein step (b) comprises incubating the sample with the protein binding agent prior to contacting the sample with the lipid binding agent.

3. The method of claim 2, wherein the sample and the protein binding agent are incubated for at least 1 hour prior to contacting the sample with the lipid binding agent.

4. The method of claim 1, wherein step (b) comprises incubating the sample with the lipid binding agent prior to contacting the sample with the protein binding agent.

5. The method of any one of claims 1-4, wherein the protein binding agent is labeled with the acceptor moiety and the lipid binding agent the fluorophore donor.

6. The method of any one of claims 1-5, wherein the acceptor moiety is a fluorophore acceptor or a fluorescence quencher.

7. The method of any one of claims 1-6, wherein the lipidated protein is a lipidated apolipoprotein.

8. The method of claim 7, wherein the lipidated apolipoprotein is selected from the group consisting of apolipoprotein A-I (ApoA-I), apolipoprotein B (ApoB), apolipoprotein D (ApoD), an apolipoprotein E (ApoE), and apolipoprotein J (ApoJ).

9. The method of claim 8, wherein the lipidated apolipoprotein is an ApoE.

10. The method of claim 8, wherein the ApoE is ApoE2, ApoE3, or ApoE4.

11. The method of any one of claims 1-10, wherein the protein binding agent is a protein, a nucleic acid, or a small molecule.

12. The method of any one of claims 1-11, wherein the protein binding agent is an antibody or an antigen binding fragment thereof.

13. The method of any one of claims 7-12, wherein the lipidated apolipoprotein is an ApoE and the protein binding agent is an anti-ApoE antibody or an antigen binding fragment thereof.

14. The method of any one of claims 7-12, wherein the lipidated apolipoprotein is ApoE2 and the protein binding agent is an anti-ApoE2 antibody or an antigen binding fragment thereof.

15. The method of any one of claims 7-12, wherein the lipidated apolipoprotein is ApoE3 and the protein binding agent is an anti-ApoE3 antibody or an antigen binding fragment thereof.

16. The method of any one of claims 7-12, wherein the lipidated apolipoprotein is ApoE4 and the protein binding agent is an anti-ApoE4 antibody or an antigen binding fragment thereof.

17. The method of any one of claims 13-16, wherein the anti-ApoE antibody or antigen binding fragment thereof is the antibody produced by clone EPR19392, clone E6D7, or clone WUE-4, or an antigen binding fragment thereof.

18. The method of any one of claims 1-17 wherein the lipid binding agent is a lipophilic fluorescent dye.

19. The method of claim 18, wherein the lipophilic fluorescent dye is selected from the group consisting of a lipophilic carbocyanine dye, a lipophilic aminostyryl dye, a PKH dye, and a lipophilic styryl dye.

20. The method of claim 19, wherein the lipophilic carbocyanine dye is selected from the group consisting of Dil, DiO, DiD, and DiR.

21. The method of claim 19, wherein the lipophilic aminostyryl compound is DiA or an analog thereof.

22. The method of claim 19, wherein the PKH dye is PKH2, PKH26, or PKH67.

23. The method of claim 19, wherein the lipophilic styryl dye is FM® 1-43, FM® 1-43FX, FM® 2-10, FM® 4-64, FM® 4-64FX, or FM® 5-95.

24. The method of any one of claims 1-17, wherein the lipid binding agent comprises a fluorophore or a fluorescent quencher.

25. The method of any one of claims 1-24, wherein the fluorophore donor is a lanthanide metal.

26. The method of claim 25, wherein the lanthanide metal is europium, terbium, or samarium.

27. The method of any one of claims 1-26, wherein the sample is a cell culture.

28. The method of any one of claims 1-26, wherein the sample is a biological sample obtained from a subject.

29. The method of claim 28, wherein the biological sample comprises blood, serum, plasma, or cerebral spinal fluid (CSF).

30. The method of claim 28 or claim 29, wherein the subject has a neurological disorder.

31. The method of claim 30, wherein the neurological disorder is a neurodegenerative disease selected from the group consisting of Alzheimer's disease (AD), multiple sclerosis (MS), and Parkinson's disease (PD).

32. The method of claim 30, wherein the neurological disorder is a neurological injury selected from the group consisting of a traumatic brain injury (TBI), a spinal cord injury (SCI), and a stroke.

33. The method of claim 30, wherein the neurological disorder is selected from the group consisting of epilepsy, dementia, and meningoencephalitis.

34. A method for treating a subject having a neurological disorder, the method comprising:
(a) detecting the level of the lipidated protein in the sample according to the method of any one of claims 1-33;
(b) comparing the level of the lipidated protein in the sample to a reference level; and
(c) administering a treatment if the level of the lipidated protein in the sample is equal to or lower than the reference level.

35. The method of claim 34, wherein the treatment is selected from the group consisting of a lipid modifying agent, an immunosuppressive therapy, a cholinesterase inhibitor, an anti-seizure therapy, an anti-inflammatory therapy, dopamine, a dopamine agonist, an antibiotic, an antiviral therapy, an apolipoprotein targeted therapy, or a combination thereof.

36. The method of claim 35, wherein the lipid modifying agent is an ABCA1 agonist or a nuclear receptor agonist.

37. The method of claim 36, wherein the ABCA1 agonist is an antisense oligonucleotide or a peptide.

38. The method of claim 37, wherein the antisense oligonucleotide is an antisense oligonucleotide targeting miR-33 or an antisense oligonucleotide targeting ARF6.

39. The method of claim 37, wherein the peptide is selected from the group consisting of CS-6253, Ac-hE18A-NH2, and 4F.

40. The method of claim 36, wherein the nuclear receptor agonist is a LXR agonist or a RXR agonist.

41. The method of claim 40, wherein the LXR agonist is TO901317 or GW3965.

42. The method of claim 40, wherein the RXR agonist is bexarotene, LG100268, SPF1, or SPF2.

43. The method of claim 35, wherein the apolipoprotein targeted therapy is an ApoE targeted therapy selected from the group consisting of an anti-ApoE antibody or an antigen binding fragment thereof, a nucleic acid that inhibits expression of ApoE, an agent that blocks the activity of and/or lowers the level of ApoE, an ApoE4 structure corrector, an anti-ApoE4 immunotherapy, an agent that enhances expression of ApoE2, and agent that inhibits expression of ApoE4.

44. The method of claim 43, wherein the ApoE4 structure corrector is PH002, GIND105, or GIND-25.

45. The method of claim 43, wherein the anti-ApoE4 immunotherapy is the anti-human ApoE4 antibody HAE-4.

46. The method of claim 43, wherein the agent that enhances expression of ApoE2 is an AAV-expressing human ApoE2.

47. The method of claim 43, wherein the agent that inhibits expression of ApoE4 is an antisense oligonucleotide targeting ApoE4.

48. The method of claim 43, wherein the anti-ApoE antibody or an antigen binding fragment thereof binds to unlipidated ApoE.

49. The method of claim 43, wherein the nucleic acid that inhibits expression of ApoE is an antisense oligonucleotide targeting ApoE or an siRNA targeting ApoE.

50. The method of claim 43, wherein the agent that blocks the activity of and/or lowers the level of ApoE blocks the activity of and/or lowers the level of unlipidated ApoE.

51. The method of claim 34, wherein the neurological disorder is Alzheimer's disease.

52. The method of claim 51, wherein the lipidated protein is ApoE4.

53. The method of claim 51 or 52, wherein the treatment is an anti-amyloid beta antibody.

54. The method of claim 53, wherein anti-amyloid beta antibody is aducanumab, bapineuzumab, gantenerumab, solanezumab, donanemab, or lecanemab.
